(19) United States
(12) Patent Application Publication
(10) Pub. No.: US 2013/0079527 A1
(43) Pub. Date: Mar. 28, 2013

(54) Method for Separating off Tryptophan

(71) Applicants: Christian Kessler, Aschaffenburg (DE); Wilfried Blümke, Schoneck (DE); Hermann Lotter, Altenstadt (DE); Joachim Pohlisch, Gelnhausen (DE)

(72) Inventors: Christian Kessler, Aschaffenburg (DE); Wilfried Blümke, Schoneck (DE); Hermann Lotter, Altenstadt (DE); Joachim Pohlisch, Gelnhausen (DE)

(73) Assignee: Evonik Degussa GmbH, Essen (DE)

(21) Appl. No.: 13/697,475

(22) PCT Filed: Apr. 28, 2011
(86) PCT No.: PCT/EP11/56718

§ 371 Date: Dec. 6, 2012

(30) Foreign Application Priority Data
May 12, 2010 (DE) 102010028916.7

(51) Int. Cl. C07D 209/20

(52) U.S. Cl. CPC C07D 209/20
USPC 548/497; 210/198.2

(57) ABSTRACT

The invention relates to a method for separating off tryptophan from aqueous mixtures of matter, in particular fermentation broths that are already partially processed, using simulated countercurrent chromatography or simulated moving bed (SMB) chromatography, and a device for carrying out the method.

The invention relates to a method for separating off tryptophan from aqueous mixtures of matter, in particular fermentation broths that have already been partially processed, using simulated countercurrent chromatography or simulated moving bed (SMB) chromatography, and a device for carrying out the method.

### PRIOR ART

Tryptophan is generally produced by fermentation, i.e. using microorganisms. This applies particularly to its biologically utilizable L form. After the fermentation, the biomass is killed, separated off and the liquid supernatant further processed. At the end of the purification chain there is generally a crystallization in which tryptophan is obtained as a high-purity solid by separation from the liquid supernatant, which is termed the mother liquor. This is saturated in tryptophan and additionally contains further salts, other amino acids and also other organic components not defined in more detail which are formed during the fermentation.

If, after the fermentation, the likewise aromatic amino acids phenylalanine and tyrosine are present in non-negligible concentrations, separating them off from tryptophan from the mother liquor is difficult owing to the similar physicochemical properties, and so the mother liquor must be frequently discarded.

U.S. Pat. No. 5,300,653 describes separating off aromatic amino acids from aqueous solutions using cation-exchange chromatography, without specifying a defined form of the procedure. The two aromatic amino acids tryptophan and phenylalanine were not able to be separated using the method described there.

In a publication by Ribeiro et al. in Bioprocess Engineering 12 (1995) 95-102, separating off tryptophan from mixtures of the aliphatic amino acid serine and indole by selective adsorption of tryptophan onto various activated carbons and absorbents of organic polymers at pH 8.0 is described. Inter alia, the polymer XAD-7 (Rohm & Haas, Philadelphia, USA) was used. The starting material was a solution of serine and indole from which tryptophan was produced using immobilized microorganisms. The separation mixture did not contain any further amino acids or amino acid-like components, apart from these three substances. The mixture composition, in addition, in contrast to starting media produced by fermentation, is not subject to fluctuations. The separation proceeded discontinuously. As a non-organic water-based desorption medium, an NaOH solution (0.05 M) and also an HCl solution (0.1 M) are described. Desorption using pure water is not mentioned there. Tryptophan could only be desorbed from another adsorber (XAD-4, Rohm & Haas, Philadelphia, USA) by addition of an organic solvent, for example methanol or isopropanol.

In a publication by Wu et al. in Industrial and Engineering Chemistry Research 37 (1998) 4023-4035, the separation of a defined two-component mixture of the aromatic amino acids tryptophan and phenylalanine without a temperature gradient in water is described. The adsorber used was a PVP resin (poly-4-vinylpyridine, Reillex HP polymer, Vertellus Specialties Inc, Indianapolis, USA). The desorbent used was water. The separation proceeded in a similar manner to the known 4-Zone Simulated Moving Bed Method using a closed internal liquid circuit. The separation task is simplified to a significant extent by the defined two-component mixture, since therein, in contrast to starting solutions produced by fermentation, no side reactions with non-defined other compounds can occur.

The adsorption of amino acids onto neutral polymeric adsorbents has additionally been described by Doulia et al. in Journal of Chemical Technology and Biotechnology 76 (2001) 83-89. It was observed here that the aromatic amino acids tryptophan and phenylalanine bind most strongly to the two adsorber materials XAD-2 and XAD-4 (Rohm & Haas, Philadelphia, USA). Increasing the ionic strength in the solution led to an increased adsorption. However, no details on desorption were given.

EP 1 106 602 B1 discloses separating off a basic amino acid (L-lysine) using the method of simulated countercurrent chromatography from solutions that contain L-lysine and further impurities.

The series-connected chromatographic columns are packed with a strong cation exchanger.

However, cation exchangers are unsuitable for separating off tryptophan if the solutions additionally contain phenylalanine and/or tyrosine, since these interact with an ion exchanger in a comparable manner.

In U.S. Pat. No. 5,071,560 the selective adsorption of phenylalanine onto the neutral polymer XAD-7 and also desorption thereof using water, alcohol, ketones or esters is described. In this method, the phenylalanine that is to be adsorbed is separated from other components that do not interact with the adsorber and detached again from the adsorber by desorption. A fermentation broth is used which, in addition to phenylalanine, substantially contains salts, lactic acid, and other amino acids that are not defined in more detail.

The rudiments of the SMB method were described for the first time in U.S. Pat. No. 2,985,589 (1961). In recent years a multiplicity of different process variants have been tested. Overviews may be found, for example, in “Fundamentals of Preparative and Nonlinear Chromatography” by Guiochon et al. (Academic Press 2006, New York, USA) or in Seidel-Morgenstern et al. Chemical Engineering and Technology 31 (2008) 826-837.

An SMB approach based on changing solvent compositions (solvent gradient) with three active zones and one displaced region for compensating for gradient fluctuations was described, for example, by L. C. Keβler et al. in Journal of Chromatography A 1176 (2007)69-78 with reference to the purification of antibodies and proteins. A decisive feature of the method is the use of differing salt concentrations for achieving differing interaction strengths. The desorption media used were sodium chloride solutions of differing concentrations.

The object of the invention is to provide a method for separating off tryptophan from aqueous mixtures of matter, in particular the mother liquors occurring after the crystallization of tryptophan from fermentation broths, and hence to increase the yield of the tryptophan fermentation.

The invention relates to a method for separating off dissolved tryptophan from an aqueous mixture of matter, in particular an aqueous mixture of matter containing further aromatic amino acids, with the aid of a variant of simulated countercurrent chromatography or SMB chromatography in which, in a separation section having a column arrangement consisting of more than one series-connected columns packed with an organic polymer suitable as adsorbent, which column arrangement is subdivided into a plurality of, preferably three, functional zones,
a) the dissolved tryptophan-containing mixture of matter and water as desorbent are continuously fed to the column arrangement at separate points and
b) at a point situated between these feeds, the tryptophan-enriched extract stream, and at a further point situated upstream of the feed of tryptophan-containing mixture of matter, a raffinate stream containing further compounds from the mixture of matter used, are taken off separately, optionally processed further and preferably
c) the columns that are loaded with non-desorbed compounds from the mixture of matter are cleaned by extraction of said compounds.

Enriched means that the extract stream contains tryptophan in a higher purity than in the mixture of matter that is fed in.

In one embodiment, the mixture of matter is in particular the fermentation broths occurring after the fermentation of a tryptophan-producing microorganism, which fermentation broths, in addition to further impurities, also contain phenylalanine and/or tyrosine, and from which, preferably, the biomass has been separated off in advance.

Preferably, the method according to the invention is used subsequently to the crystallization in which tryptophan is obtained as a high-purity solid and, therefrom, the liquid supernatant, termed the mother liquor, is separated off. This is saturated in tryptophan and additionally still contains salts, other amino acids, such as phenylalanine and/or tyrosine, for example, and other compounds that were formed during the fermentation which are not defined more clearly and act as impurities. This mother liquor, in a preferred variant, is separated by the claimed process into
a) a product stream (extract) in which the predominant amount of the tryptophan present in the mother liquor occurs, and which is simultaneously greatly depleted in interfering substances, and
b) a waste stream (raffinate), in which the further undesired mother liquor constituents are present.

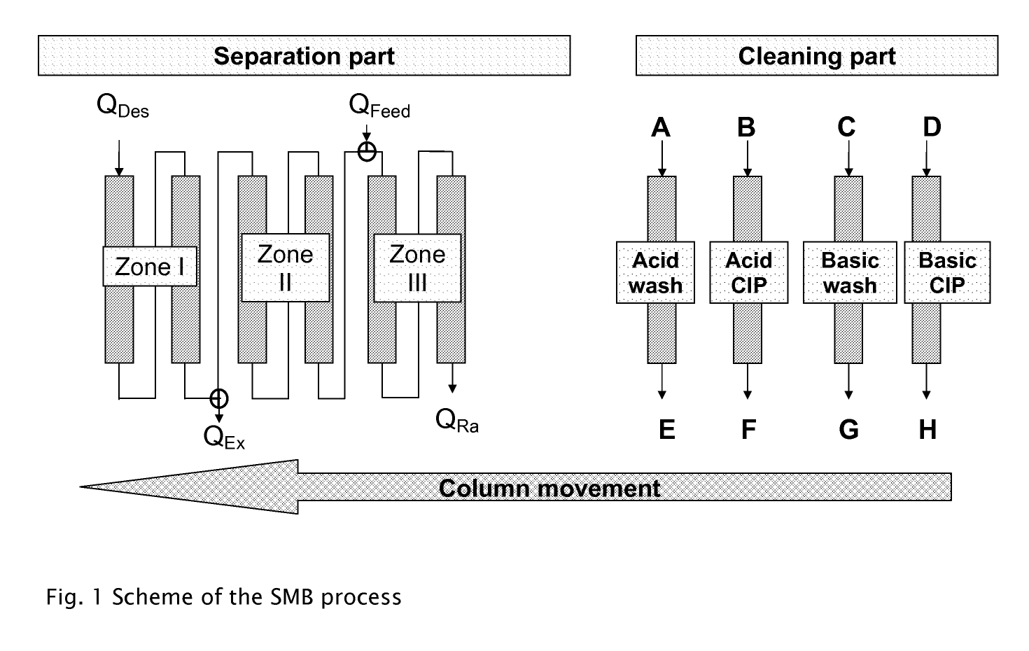

In FIG. 1 the process scheme is reproduced.

The product stream obtained is then reintroduced into the main stream of the fermentation broth work-up process upstream of the crystallization step, optionally after a concentration by evaporation and thus further processed. If the stream used is a cell-free fermentation solution, the product stream can be further processed directly as such. The yield of the production of tryptophan by fermentation is increased thereby. This is part of the invention.

The method according to the invention can also replace the described crystallization.

The separation proceeds using an organic polymer that is characterized by a medium polarity. As could be shown in the present invention, this material is able to separate off tryptophan virtually completely from the aromatic amino acids phenylalanine and tyrosine and simultaneously greatly deplete other impurities. These also include, in particular, UV-active byproducts (“UV-byproducts”), that elute, in a standard analytical method for impurities as specified in European Pharmacopoeia 6.3, either before (“UV-BP before”) or after (“UV-BP after”) tryptophan.

Suitable adsorbers are particularly nonionic polymeric adsorbents which adsorb tryptophan reversibly in such a manner that their interaction with the tryptophan is greater than that with the impurities. The relatively strongly retained tryptophan can be desorbed again with water in a preferred temperature range from 20 to approximately 80° C., wherein a higher temperature leads to a more rapid desorption and thereby to a lower retention of tryptophan. A majority of the organic impurities likewise contained in the fermentation broths used such as, for example, fragments of proteins of microbial origin or byproducts such as phenylalanine or tyrosine do not interact or interact much more weakly than tryptophan with the adsorbent during the separation step proceeding according to the invention, in such a manner that they may be found in the pass-through or in the raffinate. A few impurities are likewise adsorbed and also remain adhering to the adsorber during the desorption with water. They are desorbed in a subsequent cleaning step using alkaline or acidic solutions. Subsequently the columns thus cleaned are again connected into the separation process. Suitable adsorbents comprise polymers from the group of materials containing acrylic/methacrylic group and polystyrene-based polymers.

Preferred adsorbents are acrylic polymers such as, e.g., XAD7, XAD7HP® (Rohm & Haas) or HP2MG® (Diaion).

The adsorbents which are particularly suitable comprise
a) a backbone of acrylates, preferably methacrylates, acrylic acid and derivatives thereof
b) an aliphatic crosslinking, preferably by polyfunctional monomers
c) a dipole moment that results from the two abovementioned properties, which dipole moment is higher than in ST-DVB-based materials
d) a macroreticular pore structure
e) a suitably high surface area
f) the absence of interaction via ion exchange

The separation system is not burdened with organic solvents, since, according to the invention, for the separation only water is required, in particular demineralized water, which is fed in at a temperature of from 20 to ˜98° C., preferably 60 to 70° C. This makes possible a simple process procedure and avoids an introduction of additional foreign matter. The implementation in terms of apparatus proceeds using a variant of the “Simulated Moving Bed” process that is shown in FIG. 1.

The columns are connected to one another using valve structures which are available on the market and, in one implementation form, after expiry of a certain time (the “switching time”), using this circuit, are circulated simultaneously. In the present case, this is achieved by a central switching valve from Knauer (Knauer Wissenschaftliche Gerätebau GmbH, Berlin, Germany). The method, however, can also be implemented using other valve structures, for example using suitably connected two-way valves. The solids countercurrent in this method is simulated by a position change in the feeds and outlets. In this case, non-simultaneous further connection of the feeds and outlets is also possible according to U.S. Pat. No. 6,136,198. A further possibility is carrying out sequentially the steps described hereinafter, for instance according to the sequential SMB principle (S, Baudouin and X. Lancrenon, Industries Alimentaires et Agricoles, 120, 2003, pages 42-48). In principle, the basic principle of the method according to the invention may be adapted to all already known variants of continuous and discontinuous chromatography and thereby equips these with new functionalities.

During the SMB operation, the liquid stream passes through a plurality of fixed-bed columns packed with adsorbent. The device according to the invention is subdivided into three functional zones by the continuous addition or removal of feed, desorbent, extract and raffinate streams, as shown in FIG. 1. Each individual one of these zones in this case adopts a specific separation or work-up function. It is advantageous for the purposes of the invention, when employed with mixtures that are to be separated and are not defined exactly, to use an open liquid circuit (“open loop”). Each functional zone contains in each case one to a plurality of chromatographic columns.

In addition, preferably with addition of a fourth zone which is upstream of the raffinate withdrawal point, a closed liquid circuit can also be implemented.

In the operation preferably open according to the invention of the SMB method, there exist in general three internal (Q_{I}, Q_{II} and Q_{III}) and four external (Q_{Feed}, Q_{Des}, Q_{Ex} and Q_{Ra}) material flow rates that are linked to one another by means of the following balance equations

Q_{I}=Q_{Des}

Q_{II}=Q_{I}−Q_{Ex}

Q_{III}=Q_{II}+Q_{Feed}=Q_{Ra}.

For establishing the operating point, the mass flow rates or volumetric flow rates and also the cycle time tₛ must be specified in such a manner that the separation task is solved and thereby an economically optimum operation is achieved with compliance with the preset product units. The cycle time tₛ determines in this case the “velocity” of the apparent solids countercurrent and thereby decisively determines the separation success. Establishing the streams can be implemented according to the prior art in various ways, such as described in “Fundamentals of Preparative and Nonlinear Chromatography” by Guiochon et al. (Academic Press 2006, New York, USA).

The countercurrent generated in the SMB process is utilized in order to separate tryptophan from a mixed fraction of pollutants. The basis is the respectively differing migration rates of the compounds which are used to direct tryptophan and the mixed fraction of the pollutants (“neutral waste”) to two different outlets (FIG. 1). From the outlet designated “extract”, during the entire process the tryptophan-containing product stream may be taken off, and the interfering substances are removed with the stream designated “raffinate”. In addition, a continuous addition of temperature-controlled water proceeds via the “desorbent” inlet. The separation success is determined by a suitable selection of the flow rates of the feeds and outlets and also of the switching time.

According to the invention, in the separation section consisting of three zones, a temperature gradient is maintained. The feed of the temperature-controlled water proceeds at a temperature generally 10 to 40° C. higher than that of the solution that is to be separated (separation mixture) and introduced via the feed entrance. Feed temperatures of the desorbent of at least 60° C. and 45° C. in the separation mixture have proved advantageous.

The water used as desorbent is preferably fully desalted. However, it is also possible to use water having a salt content, preferably from 0.01 to 10% by weight, in particular 0.01 to 3% by weight. Tryptophan is also separated off under these conditions. In this case, however, it must be accepted that the extract stream is burdened with this salt.

Aqueous mixtures of matter, in particular solutions, are used that contain 0.1 to 39 g/l, in particular 0.5 to 38 g/l, particularly preferably 10 to 18 g/l, of tryptophan.

The content is dependent, in particular, on whether mother liquors or other non-purified tryptophan solutions are to be worked up and at what pH these are present.

The pH of the mixtures or solutions used ranges from 2 to 9, in particular from 2.5 to 7, and is particularly 5.8.

In the method according to the invention, in parallel to the separation process, the columns that are not required for the separation in regular passage are treated in a cleaning section (“cleaning part”) with various agents in order to remove from the adsorbent impurities binding strongly thereto and also poorly soluble impurities.

The columns for this purpose are first treated with a suitable aqueous basic cleaning agent. Suitable cleaning agents, without being restricted thereto, are, in particular, sodium hydroxide solutions at concentrations from 0.05 to 1 M. Furthermore, other solutions can be used which, according to the prior art, are able to free the adsorbent from strongly binding impurities. By way of example, without being restricted thereto, water-miscible organic solvents can be mentioned here such as, e.g., ethanol, methanol, acetone or isopropanol.

In a further parallel process step, the columns already treated with the first cleaning agent are flushed with a suitable medium. This is, in particular, desalted water, but other aqueous solutions having a buffer action can also be used. In the case of a suitable cleaning agent, the flushing step before the second cleaning can also be dispensed with.

In a further parallel step, the columns that are already treated with the first cleaning agent and are optionally flushed are contacted with a second cleaning medium. A particularly suitable cleaning medium here is an acidic aqueous solution, for example 0.01 to 0.1, in particular 0.05 M sulfuric acid. Furthermore, other cleaning media can also be used which, according to the prior art, are able to dissolve sparingly soluble substances better in a neutral or basic environment.

In a further parallel step, the columns that are already treated with the two cleaning agents are either again flushed, or flushed for the first time with a suitable aqueous medium. This is, in particular, desalted water, but other aqueous solutions can also be used.

In a preferred form of the invention, all cleaning steps are carried out continuously and in parallel to the running separation.

In a further implementation form, individual partial steps can be omitted.

In a further implementation form, all, or only selected, cleaning steps can also be carried out in a manner decoupled from the separation. Thus it is possible, for example, to carry out the cleaning in only a daily rhythm or a rhythm coupled to a defined separation running time.

The invention also relates to a device for separating off desired organic compounds from an aqueous mixture of matter with the aid of simulated countercurrent chromatography or SMB chromatography, in which one column circuit consisting of more than one series-connected, adsorbent-packed columns and which is subdivided into three functional zones in which the following tasks are implemented

Addition of the feed stream,
Withdrawal of the eluate stream,
Withdrawal of the raffinate stream,
Addition of the desorbent,
and in which, between zones I and III, a temperature gradient of 10 to 40° C., in particular 15 to 25° C. according to FIG. 1, is established caused by the differing feed temperatures in the desorbent and feed. The temperature gradient promotes the separation and decreases the consumption of desorbent. However, it is not a necessary condition for the separation to succeed.

This structure differs from the known devices in that, in the separation section, chromatography columns are arranged in a three-zone arrangement with an open liquid circuit and a temperature gradient and this system part is coupled to a cleaning section (“cleaning part”) which contains columns of the same adsorbent pack which are cleaned by removing the adherent impurities in alternation with those from the separation section.

The number of columns in the zones generally ranges from more than one to 32, in particular 16. Each zone contains at least one column. The total number thereof and distribution over the zones can, in adaptation to the temperature, the tryptophan concentration, the desired migration rate and the purity of the product, be determined by standard experiments. The number of columns is further affected also by the type of the process procedure and the implementation of the SMB method.

Using the method according to the invention, it was possible in laboratory experiments, starting from mother liquors having tryptophan concentrations between 9 and 22 g/kg of mother liquor and purities between 7 and 15% (based on the total dry mass), to achieve product streams having a mean purity of greater than 85%, a mean tryptophan yield of better than 85%, and a tryptophan concentration from 4 to 5 g/kg in the extract.

It is possible thereby to separate off the tryptophan amounts present in the mother liquor to give a high percentage in purified form and to recirculate them in an economical manner into the fermentation broth work-up process for improving the overall yield.

### DESCRIPTION OF FIG. 1

FIG. 1 reproduces a schematic structure and the column arrangement of the SMB process used according to the invention.

The arrangement is divided into a separation part and a cleaning part.

In the separation part, for example, the desorbent stream Q_{Des} is fed in at 60° C. and the aqueous mixture Q_{Feed} that is to be separated is fed in at 45° C. and the raffinate Q_{Ra} and the extract Q_{Ex} in which the tryptophan is present in separated-off form are withdrawn.

In the cleaning part, for example at positions A and B, water is fed, at position B, 0.05 M sulfuric acid is fed and at position D, 0.5 M NaOH is fed.

After the passage through the columns that are to be cleaned, at position E, consumed sulfuric acid exits, and at position F a solution of more lightly bound, water-soluble impurities exits, at position G consumed NaOH exits, and at position H a solution having the strongly bound impurities exits. (CIP=Cleaning In Place).

### EXAMPLES

### 1. Method Parameters

Adsorber used: Amberlite XAD-7 HP (Rohm & Haas)
Column dimensions (d×L): 1.5×15 cm
SMB system: Knauer CSEP 916 (Knauer Wissenschaftliche Gerätebau GmbH, Berlin, Germany)
Pumps: Watson-Marlow U101 (Wilmington, Mass., USA)
Mixture to be separated: crystallization mother liquor:

| | Concentration range |
|---|---|
| Material | [g/kg] |
| Tryptophan | 12-22 |
| Tyrosine | 1.2-2.1 |
| Phenylalanine | 1.1-4.8 |
| Further | 220-260 |
| byproducts | |

The further parameters are independent of said parameters such as, e.g., the switching times in question which were between 8 and 14 minutes. However, there is no reason to restrict these. The flow rates used in the laboratory system were between 0.07 and 0.33 kg/h, without being restricted thereto, limited by the peristaltic pumps used. Here also, any pump type can be used which is able to deliver the flow rates required for the throughput to be achieved and ensuring a separation.

The temperatures used were between room temperature (25° C.) and 60° C., a desorbent temperature of at least 60° C., but not higher than 80° C., and 45° C. for the remaining streams and columns proved advantageous.

### 2. Use Examples

### 2.1 Determination of the Adsorption Behavior

In the context of a measurement campaign, the adsorbent and desorption behavior of tryptophan, phenylalanine and tyrosine were studied using one of the columns described in section 1. For this purpose 100 μL of the sample were injected into a water stream flowing through the column and the exit time of the amino acids was detected at the outlet. It was found that tryptophan, among the aromatic amino acids, is retained most strongly both in a neutral, slightly acidic, and also in an acidic environment. Increasing the temperature led in the case of tryptophan to a shortening of the retention time:
Retention Time for Tryptophan at pH 5.9 (in Minutes)

| | 45° C. | 60° C. |
|---|---|---|
| 5 g/kg | 37.3 | 31.9 |
| 10 g/kg | 37.1 | 31.1 |
| 17 g/kg | — | 33.5 |

Retention Times for Phenylalanine at pH 5.9

| | 45° C. | 60° C. |
|---|---|---|
| 5 g/kg | 15.6 | 15.3 |
| 10 g/kg | 15.2 | 15.1 |

The retention times for tyrosine were at the same level as for phenylalanine. At a pH of 2.5, 45° C. and a concentration of 10 g/kg, the retention time for tryptophan was 39.1 minutes, for phenylalanine under the same conditions 15.8 minutes. Separation is possible therefore even at an acidic pH.

### 2.2 First SMB Experimental Campaign

Here, three operating points were experimentally examined. The feed materials used were various batches of tryptophan-containing process mother liquor and also the solutions described in section 1. The conditions were:

| | Q_{Des} | Q_{Feed} | Q_{Ex} | Q_{Ra} | t_{S} |
|---|---|---|---|---|---|
| Operating | 5.49 g/min | 1.18 g/min | 3.21 g/min | 3.47 g/min | 14.0 min |
| point 1 | | | | | |
| Operating | 5.74 g/min | 1.26 g/min | 3.30 g/min | 3.69 g/min | 12.9 min |
| point 2 | | | | | |
| Operating | 5.59 g/min | 1.27 g/min | 3.26 g/min | 3.60 g/min | 12.75 min |
| point 3 | | | | | |

The temperature of the desorbent was 60° C., that of the columns and the other solutions 45° C. The results of the experiments were:

| | TRP | | | | | |
|---|---|---|---|---|---|---|
| | purity | | | | | |
| | (dry | TRP | Removal | Removal | Removal | Removal |
| | mass) | yield | of | of | of UV BP | of UV BP |
| | [%] | [%] | PHE [%] | TYR [%] | before [%] | after [%] |
| Operating | 87.1 | 37 | 100 | 100 | n.d. | n.d. |
| point 1 | | | | | | |
| Operating | 82.2 | 76 | 100 | 100 | 92.4 | 34.9 |
| point 2 | | | | | | |
| Operating | 87.6 | 85 | 99.1 | 96.1 | 93.5 | 41.8 |
| point 3 | | | | | | |

### 2.3 12-Day Experiment

In a further campaign, the long-term stability was evaluated. Over an experimental time period of 12 days, the process was run with the following parameters:

| Q_{Des} | Q_{Feed} | Q_{Ex} | Q_{Ra} | t_{S} |
|---|---|---|---|---|
| 5.6 g/min | 1.3 g/min | 3.26 g/min | 3.60 g/min | 12.75 min |

| Q_{NaOH} | Q_{BasicWash} | Q_{H2SO4} | Q_{AcidWash} |
|---|---|---|---|
| 2.35 g/min | 1.91 g/min | 2.89 g/min | 2.02 g/min |

The feed materials used were mother liquors from the tryptophan crystallization. In the course of the process, average purities of 89.3% (based on the total dry mass) and also yields of 90.4% (based on the tryptophan mass used) could be achieved. The concentrations of phenylalanine and tyrosine could be reduced on average to 0.002 and 0.001 g/l, respectively. Recirculation to the tryptophan crystallization was able to be achieved successfully and exhibited no adverse effects.

1.-17. (canceled)

18. A method for separating off tryptophan from an aqueous mixture of matter using simulated countercurrent chromatography or SMB chromatography in which, in a separation section having a column arrangement consisting of more than one series-connected columns packed with an organic polymer suitable as adsorbent, which column arrangement is subdivided into a plurality of functional zones,
a) the tryptophan-containing mixture of matter and water as desorbent are continuously fed to the column arrangement at separate points and
b) at a point situated between these feeds, the tryptophan-enriched extract stream, and at a further point situated upstream of the feed of tryptophan-containing mixture of matter, a raffinate stream containing further compounds from the mixture of matter used, are taken off separately, the extract stream optionally processed further and optionally
c) the columns that are loaded with non-desorbed compounds from the mixture of matter are cleaned by extraction of said compounds.

19. The method as claimed in claim 18, in which the water is optionally desalted and is fed at a temperature of from 20 to 98° C.

20. The method as claimed in claim 18, in which a mixture of matter is used that contains tryptophan at a concentration from 0.1 to 39 g/l.

21. The method as claimed in claim 18, in which a mixture of matter is used that has a pH from 2.5 to 9.

22. The method as claimed in claim 18, in which a mixture of matter obtained by fermentation is used that, in addition to tryptophan, also contains phenylalanine and/or tyrosine.

23. The method as claimed in claim 22, in which the mixture of matter used is the mother liquor which occurs on crystallization of tryptophan from the fermentation broth.

24. The method as claimed in claim 18, in which the adsorbent used is a non-ionic polymer that reversibly adsorbs tryptophan and from which the tryptophan is desorbed with water in the temperature range from 10 to approximately 98° C.

25. The method as claimed in claim 18 in which adsorbents selected from the group consisting of XAD7®, XAD7HP® and HP2MG®, and combinations thereof are used.

26. The method as claimed in claim 18, in which a temperature gradient is established between the zone in which the desorbent is introduced and the zone from which the raffinate is conducted.

27. The method as claimed in claim 18, in which the separation proceeds continuously.

28. The method as claimed in claim 18, in which the separation proceeds semi-continuously.

29. The method as claimed in claim 18, in which, in parallel to separating off the tryptophan, the columns of the arrangement that are switched out of the separation, in dependence on the cycle time, are treated subsequently thereto with desorbing compounds in a cleaning section.

30. The method as claimed in claim 18, in which, in the cleaning section, the adsorbent is treated in a method comprising the following steps,
a) the columns for this purpose are first treated with a suitable basic aqueous cleaning agent, in particular sodium hydroxide solutions at concentrations from 0.05 to 1 M, optionally combined
b) in a further parallel process step optionally the columns treated according to a) are flushed with an aqueous medium selected from water which is optionally desalted, or aqueous solutions having a buffer action,
c) in a further parallel step the columns that are treated according to a) and b) are contacted with a second aqueous cleaning medium, and
d) in a further parallel step the treated columns are either optionally flushed with an aqueous medium, selected from water that is optionally desalted, or aqueous solutions that can be used as a buffer.

31. The method as claimed in claim 18, in which the extract stream containing the tryptophan is added to an aqueous mixture of matter containing tryptophan obtained by fermentation before the tryptophan is crystallized out therefrom.

32. The method as claimed in claim 30, in which all or some of the cleaning steps are carried out in an established rhythm decoupled from the separation.

33. A device for separating off desired organic compounds from an aqueous mixture of matter with the aid of simulated countercurrent chromatography or SMB chromatography, comprising one column circuit consisting of more than one series-connected, adsorbent-packed columns and which is subdivided into three functional zones that contain the following functionalities
Addition of the feed stream,
Withdrawal of the eluate stream,
Withdrawal of the raffinate stream,
Addition of the desorbent water
and in which a temperature gradient which is in the range from 10° C. to 80° C. is established between the zones I and III via a higher feed temperature of the desorbent compared to the feed stream.

34. The device as claimed in claim 33, which, in addition to the columns present in the separating section, comprises further columns in a cleaning section which are packed with the same adsorbent and, in alternation with the columns situated in the separating section for separating off the desired organic compounds, are connected, with the cycle times being maintained, into the cleaning section for removing the impurities remaining adhering to the adsorbent.

35. The process of claim 30, wherein the second aqueous cleaning medium is an acidic aqueous solution.

36. The process of claim 30, wherein the second aqueous cleaning medium is 0.01 to 0.05 M sulfuric acid.
